(19) United States
(12) Patent Application Publication
(10) Pub. No.: US 2006/0094648 A1
(43) Pub. Date: May 4, 2006

(54) Methods for treating or preventing ischemic injury

(76) Inventor: Jonathan S. Stamler, Chapel Hill, NC (US)

Correspondence Address: MINTZ LEVIN COHN FERRIS GLOVSKY & POPEO, 666 THIRD AVENUE, NEW YORK, NY, 10017, US

(21) Appl. No.: 10/521,496

(22) PCT Filed: Jul. 8, 2003
(86) PCT No.: PCT/US03/21828

§ 371 Date: Jun. 28, 2005

(30) Foreign Application Priority Data
Jul. 10, 2002 (US) 192448

(51) Int. Cl. A61K 38/18

(52) U.S. Cl. 514/12

(57) ABSTRACT

A therapeutic or prophylactic treatment method of myocardial ischemia, such as due to myocardial infarction, by administering erythropoietin, alone or in combination with other drugs, to a patient suffering from or at risk of cardiac injury, such as myocardial ischemia. The erythropoietin is administered in a concentration such that the subject's hematocrit level or production of red blood cells is not significantly affected.

### FIELD OF THE PRESENT INVENTION

The present invention generally relates to methods, preparations and pharmaceutical compositions for treating or preventing ischemic injury in mammalian subjects. More specifically, the present invention uses erythropoietins to treat myocardial ischemia or ischemia-reperfusion injury in patients in need thereof.

### DESCRIPTION OF THE RELATED ART

Ischemia occurs when the flow of blood to a region of the body is decreased or eliminated, such as during a myocardial infarction, causing damage to the tissue distal to the blockage. In the United States, approximately 1.5 million myocardial infarctions (MIs) occur each year, and mortality with acute infarction is approximately 30 percent (Pasternak, R. and Braunwald, E., Acute Myocardial Infarction, HARRISON'S PRINCIPLES OF INTERNAL MEDICINE, 13th Ed., McGraw Hill Inc., p.p. 1066-77 (1994)). Myocardial infarction occurs generally with an abrupt decrease in coronary blood flow that follows a thrombotic occlusion of a coronary artery. The occluded artery often has been narrowed previously by atherosclerosis, and the risk of recurrent nonfatal myocardial infarction persists in many patients. Ultimately, the extent of myocardial damage caused by the coronary occlusion depends upon the “territory” supplied by the affected vessel, the degree of occlusion of the vessel, the amount of blood supplied by collateral vessels to the affected tissue, and the demand for oxygen of the myocardium whose blood supply has suddenly been limited (Pasternak, R. and Braunwald, E. Acute Myocardial Infarction, HARRISON'S PRINCIPLES OF INTERNAL MEDICINE, 13th Ed., McGraw Hill Inc., p.p. 1066-77 (1994)).

In some cases, the flow of blood to a region of the body is temporarily halted and then re-established (reperfusion), resulting in ischemia-reperfusion injury. Ischemia-reperfusion injury can occur during certain surgical procedures, such as repair of aortic aneurysms and organ transplantation. Clinically, ischemia-reperfusion injury is manifested by such complications as pulnonary dysfunction, including adult respiratory distress syndrome, renal dysfunction, consumptive coagulopathies including thrombocytopenia, fibrin deposition into the microvasculature and disseminated intravascular coagulopathy, transient and permanent spinal cord injury, cardiac arrhythmias and acute ischemic events, hepatic dysfunction including acute hepatocellular damage and necrosis, gastrointestinal dysfunction including hemorrhage and/or infarction and multisystem organ dysfunction (MSOD) or acute systemic inflammatory distress syndromes (SIRS). The injury may occur in the parts of the body to which the blood supply was interrupted, or it can occur in parts fully supplied with blood during the period of ischemia.

Erythropoietin (also known as Epo, epoetin or procrit) is an acidic glycoprotein hormone of approximately 34,000 dalton molecular weight occurring in multiple forms, including alpha, beta, omega and asialo. Erythropoietin stimulates red blood cell production. It is produced in the kidney and stimulates the division and differentiation of committed erythroid precursors in the bone marrow and elsewhere. Generally, erythropoietin is present in very low concentrations in plasma when the body is in a healthy state, in which tissues receive sufficient oxygenation from the existing number of erythrocytes. This normal low concentration is enough to stimulate replacement of red blood cells that are lost normally through aging. The amount of erythropoietin in the circulation is increased under conditions such as hypoxia, when oxygen transport by blood cells in the circulation is reduced. Hypoxia may be caused by loss of large amounts of blood through hemorrhage, destruction of red blood cells by over-exposure to radiation, reduction in oxygen intake due to high altitudes or prolonged unconsciousness, or various forms of anemia or ischemia. In response to tissues undergoing hypoxic stress, erythropoietin will increase red blood cell production by stimulating the conversion of primitive precursor cells in the bone marrow into proerythroblasts which subsequently mature, synthesize hemoglobin and are released into the circulation as red blood cells. When the number of red blood cells in circulation is greater than needed for normal tissue oxygen requirements, erythropoietin in circulation is decreased.

Clinically, erythropoietin is used as a treatment for anemia associated with renal disease, cancer chemotherapy, malignancies, adult and juvenile rheumatoid arthritis, disorders of hemoglobin synthesis, prematurity, and treatment of HIV infection. Erythropoietin is primarily used to induce production of red blood cells to combat anemia. (See, e.g., Bottomley et al. (2002) Lancet Oncol. 3:145). Erythropoietin has also been suggested to be useful in controlling bleeding in patients with abnormal hemostasis. (See e.g., U.S. Pat. No. 6,274,158). Recombinant human erythropoietin (rHuEpo or epoetin α) is commercially available as EPOGEN® (epoetin alfa, recombinant human erythropoietin) (Amgen Inc., Thousand Oaks, Calif.) and as PROCRIT® (epoetin alfa, recombinant human erythropoietin) (Ortho Biotech Inc., Raritan, N.J.).

The normal ranges for hematocrit values are 37-48 percent for women and 42-52 percent for men. (See Case Records of the Massachusetts General Hospital: normal reference laboratory values. (1992) N. Eng. J. Med. 327:718). However, hematocrit values in patients with renal failure are generally below 33 percent (See Besarab et al. (1998) N. Eng. J. Med. 339: 584). Several studies have indicated that increasing hematocrit levels in hemodialysis patients from below 30 percent to 30 to 38 percent increases cardiac function and other parameters, and that the mortality rate in patients with hematocrits below 30 is higher than patients with hematocrits of 30 to 35 percent. Other studies have shown that the use of erythropoietin to increase hematocrit levels in patients with cardiovascular disease (congestive heart failure, coronary artery disease or prior myocardial infarction) to within the normal range (42 percent) results in a slight increase in adverse cardiac events (i.e., death or a first non-fatal myocardial infarction) as compared to subjects in which erythropoietin was used to increase hematocrit levels to 30 percent. (See Besarab et al. (1998) N. Eng. J. Med. 339: 584). In transgenic mice made polyglobulic (hematocrit of 80 percent) by the exogenous expression of erythropoietin, mortality due to cardiovascular complications was prevented by activation of the nitric oxide pathway. (See Ruschitzka et al. (2000) P.N.A.S. 97:11609). Thus the safety and efficacy of use of erythropoietin to increase hematocrit levels in patients with cardiovascular disease, especially those suffering from renal failure, must be further evaluated. Also unexplored are the protective and therapeutic effects of erythropoietin on the cardiovascular system independent of its hematopoietic activities.

### SUMMARY OF THE INVENTION

The present invention relates to the discovery that myocardial oxidative and/or nitrosative stress can be prevented or minimized by administration of cardioprotective factors, and thus has benefit for treating cardiovascular and other diseases. In particular, it has been found that erythropoietin is useful as a cardioprotective and cardiotherapeutic agent, and is therefore valuable in the treatment of a variety of various heart-related ailments such as myocardial infarction, ischemia-reperfusion injury, congestive heart failure, and cardiac arrest, and for cardioprotection. Erythropoietin in particular has been found to protect myocardial contractility in a subject suffering from a cardiac injury without increasing hematocrit levels. Erythropoietin has also been found to preserve cardiac beta adrenergic receptor density during cardiac injury. These discoveries provide the means to improve pharmacologically the contractility of failing or otherwise impaired hearts, and provide both endogenous and pharmacological means to improve the function of an impairment of heart rhythm or pump function.

The compositions and methods of the invention are surprisingly useful for the reduction or elimination of hypoxic/ischemic cardiac injury in vivo and ex vivo, as well as the prevention and/or treatment of cardiovascular disease in mammals in need thereof, such as humans.

In one aspect, the present invention relates to a method of treating or preventing myocardial oxidative and/or nitrosative stress in a subject by administering erythropoietin to a subject in need thereof at a concentration that does not increase the hematocrit in said subject, such that one or more myocardial cells which are the subject of said oxidative stress are protected from cell death. Myocardial oxidative stress may be caused by hypoxia, ischemia or other causes. Myocardial nitrosative stress may be caused by drugs, infection, inflammation, hypoxia, ischemia or other causes. Erythropoietin is typically administered at a concentration or for a duration that will not induce red blood cell formation or alternatively, increase the hematocrit in a subject, e.g., between about 1 pM and less than 1000 μM, including less than 900 μM, less than 700 μM, less than 500 μM, less than 300 μM, less than 100 μM, or less than 50 μM. In other embodiments, erythropoietin is administered as a function of the subject's body weight. Erythropoietin may typically be administered at a concentration of between about 1 U/kg to 10,000 U/kg of a subject's body weight, including less than 7,500 U/kg, 5,000 U/kg, 2500 U/kg, 1000 U/kg, 750 U/kg, 500 U/kg, 250 Ug/kg, 100 Ug/kg, 50 U/kg, 25 U/kg, 10 U/kg, 5 U/kg, or 1 U/kg.

In another aspect, the present invention relates to a method of treating or preventing myocardial oxidative and/or nitrosative stress in a subject by administering erythropoietin to a subject in need thereof at a concentration that does not induce red blood cell production in said subject, such that one or more myocardial cells which are the subject of said oxidative or nitrosative stress are protected from cell death.

In another aspect, the present invention relates to a method of modulating a cardioprotective signaling pathway by administering, to a subject in need of cardioprotection, erythropoietin at a concentration that does not induce red blood cell production in the subject. This concentration of erythropoietin is an amount effective to enhance or maintain the effect of the cardioprotective signaling pathway. The cardioprotective signaling pathways include MAP kinase, PI3 kinase, an insulin-responsive pathway, hormones, ischemia preconditioning, adenosine pathways, ras, JAK/STAT, nitric oxide synthase, hemoxygenase, xanthine oxidase, NADPH oxidase, cytochrome p450, cytochrome p450 reductase, oxigenases, denitrosylases, GSNO reductase, oxygen-carrying proteins, nitric oxide-carrying proteins, and carbon monoxide-carrying proteins.

In another aspect, the present invention relates to a method of treating or preventing cardiac injury caused by hypoxia or ischemia in a subject by administering erythropoietin to a subject in need thereof at a concentration that does not increase the hematocrit in the subject, such that the hypoxia or ischemic-related injury is prevented or decreased. Cardiac injury can include myocardial infarction; cardiac arrest; ischemia-reperfusion injury; congestive heart failure; cardiotoxicity; cardiac damage due to parasitic infection; fulminant cardiac amyloidosis; heart surgery; heart transplantation; and traumatic cardiac injury. The erythropoietin may be administered prior to, at the onset of, or following cardiac injury.

In another aspect, the present invention relates to a method of treating or preventing cardiac injury in a subject by administering erythropoietin to a subject in need thereof at a concentration that does not induce red blood cell production in the subject.

In another aspect, the present invention relates to a method of treating or preventing cardiac injury in a subject by administering erythropoietin to a subject in need thereof for a time period in which the hematocrit of the subject is not increased.

In another aspect, the present invention relates to a method of preventing organ damage during organ or tissue transplantation by administering to an organ donor erythropoietin at a concentration that does not increase the hematocrit in the donor prior to or concurrent with removal of the organ, such that damage caused by reperfusion of the organ or tissue is decreased or prevented. Any organ or tissue capable of being transferred by medical procedures known in the art is encompassed by the present invention.

In another aspect, the present invention relates to a method of treating heart failure in a subject by treating the subject with erythropoietin at a concentration that does not increase the hematocrit in the subject and a compound selected from the group consisting of anti-platelet drugs, anti-coagulant drugs, anti-thrombotic drugs.

In another aspect, the present invention relates to a method of treating a survivor of a myocardial infarction by administering erythropoietin at a concentration that does not increase the hematocrit in a survivor, wherein the erythropoietin is administered in a single dose within 1 hour of the myocardial infarction.

In another aspect, the present invention relates to a method of treating a survivor of a myocardial infarction by administering erythropoietin at a concentration that does not increase the hematocrit in the survivor, wherein the erythropoietin is administered for an extended period of time.

In another aspect, the present invention relates to a method of preventing or reducing the severity of ischemia-reperfusion injury in a subject at risk for ischemia-reperfusion injury by administering to the subject an amount of erythropoietin at a concentration that does not increase the hematocrit of the subject. In some embodiments of the present invention, the ischemia-reperfusion injury can be caused by surgical repair of a thoracic aortic aneurysm, a suprarenal aortic aneurysm, liver, kidney, small intestine, or pancreas transplant, hepatic and biliary surgical resections, total or partial pancreatectomy, total and partial gastrectomy, esophagectomy, colorectal surgery, vascular surgery for mesenteric vascular disease, abdominal insufflation during laparoscopic surgical procedures, blunt or penetrating trauma to the abdomen including gun shot wounds, stab wounds or penetrating wounds or blunt abdominal trauma secondary to deceleration injury or motor vehicle accidents, hemorrhagic shock due to blood loss, cardiogenic shock due to myocardial infarction or cardiac failure, neurogenic shock or anaphylaxis.

In another aspect, the present invention relates to a method of preconditioning a subject at risk for a cardiac injury due to a surgical procedure by administering erythropoietin at a concentration that does not increase the hematocrit in the subject.

In another aspect, the present invention relates to a method of preconditioning a subject at risk for a cardiac injury due to a surgical procedure by administering erythropoietin for a time period in which the hematocrit in the subject is not increased.

In another aspect, the present invention relates to a method of increasing beta-receptor density in a subject suffering from cardiac injury by administering erythropoietin at a concentration that does not increase the hematocrit in the subject.

In another aspect, the present invention relates to a method of preserving or increasing beta-receptor sensitivity in a subject suffering from cardiac injury by administering erythropoietin at a concentration that does not increase the hematocrit in the survivor, such that beta-receptor sensitivity is not substantially decreased.

In another aspect, the present invention relates to a method of preventing reduced sensitivity to one or more cardiostimulatory compounds in a subject suffering from or at risk of a cardiac injury by administering erythropoietin at a concentration that does not increase the hematocrit in the subject such that the patients' reaction to said one or more cardiostimulatory compounds is not reduced over time.

In another aspect, the present invention relates to a method of increasing beta-receptor density in a subject suffering from cardiac injury by administering erythropoietin for a time period in which the hematocrit in the subject is not increased.

These and other objects of the present invention will be apparent from the detailed description of the invention provided below.

### BRIEF DESCRIPTION OF THE DRAWINGS

FIG. 1 depicts the protection of cardiac contractility when erythropoietin (EPO) is administered 5 minutes after suture ligation of the large branch of the left circumflex artery (LCx). MI only, LCx ligation alone; EPO, 5000 U/kg of EPO administered following LCx ligation. Basal contractility, at rest; Iso-Low, Isoproterenol 0.033 μg/kg/min; Iso-High, Isoproterenol 0.1 μg/kg/min.

FIG. 2 depicts the protection of cardiac contractility when erythropoietin (EPO) is administered 24 hous prior to suture ligation of the large branch of the left circumflex artery (LCx). MI only, LCx ligation alone; EPO, 5000 U/kg of EPO administered 24 hous prior to LCx ligation. Basal contractility, at rest; Iso-Low, Isoproterenol 0.033 μg/kg/min; Iso-High, Isoproterenol 0.1 μg/kg/min.

FIG. 3 depicts a comparison of the hematocrit data from animals receiving systemic normal saline (Control) versus Erythropoietin (EPO 5,000 U/kg).

FIG. 4 depicts a comparison of left ventricular B-receptor density in 3 groups: LCx ligation alone (MI Only); Administration of erythropoietin (5000 U/kg) followed by LCx ligation 24 hous later (EPO); and untreated group (Sham).

### DETAILED DESCRIPTION OF THE INVENTION

The features and other details of the invention will now be more particularly described with reference to the accompanying drawings and pointed out in the claims. It will be understood that particular embodiments described herein are shown by way of illustration and not as limitations of the invention. The principal features of this invention can be employed in various embodiments without departing from the scope of the invention. All parts and percentages are by weight unless otherwise specified.

Definitions

For convenience, certain terms used in the specification, examples, and appended claims are collected here. Unless otherwise defined, all technical and scientific terms used herein have the same meaning as commonly understood by one of ordinary skill in the art to which this invention pertains. However, to the extent that these definitions vary from meanings circulating within the art, the definitions below are to control.

“Erythropoietin” includes any variants, fragments, conjugates, derivatives, and mutants of the erythropoietin protein, produced by natural, recombinant or synthetic means.

“Ischemia” includes the decrease or cessation of myocardial blood flow.

“Hypoxia” includes the deficiency in the amount of oxygen reaching body tissues.

“Hypoxia or ischemic-related injury” includes cardiac injury.

“Reperfusion” includes the restoration of blood flow to an organ or tissue that has had its blood supply cut off, as after a heart attack or stroke.

“Oxidative stress” includes conditions that occur when there is an excess of free radicals, a decrease in antioxidant levels, or both.

“Nitrosative stress” includes impetus for NO or NO₂ group attachment to proteins, nucleic acids or other biological molecules. Nitrosative stress is distinct from oxidative stress and can occur under anaerobic conditions. Nitrosative stress can be caused by an increase in nitrosation or nitrosating agents, or a decrease in anti-nitrosants, or a combination of these factors.

“Cardiac stunning” includes cardiac contractile dysfunction, such as due to surgical procedures, and may include troponin-I dysfunction.

“Necrosis” includes the death of cells or tissues through injury or disease, particularly in a localized area of the body such as the myocardium.

“Apoptosis” refers to programmed cell death.

“Sensitivity” refers in part to the efficacy of a treatment.

“Anti-arrhythmic compounds” include any compounds useful in treating an irregular or abnormal heartbeat.

“Contractility enhancing compounds” include any compounds that increase myocardial contraction.

“Beta blockers” include agents such as atenolol, metoprolol, and propranolol, which act as competitive antagonists at the adrenergic beta receptors. Such agents also include those more selective for the cardiac (beta-1) receptors which allows for decreased systemic side effects. Beta blockers reduce the symptoms connected with hypertension, cardiac arrhythmias, migraine headaches, and other disorders related to the sympathetic nervous system. Beta blockers also are sometimes given after heart attacks to stabilize the heartbeat. Within the sympathetic nervous system, beta-adrenergic receptors are located mainly in the heart, lungs, kidneys, and blood vessels. Beta blockers compete with the nerve-stimulating hormone epinephrine for these receptor sites and thus interfere with the action of epinephrine, lowering blood pressure and heart rate, stopping arrhythmias, and preventing migraine headaches.

“Cardiac injury” includes any chronic or acute pathological event involving the heart and/or associated tissue (e.g., the pericardium, aorta and other associated blood vessels), including ischemia-reperfusion injury; congestive heart failure; cardiac arrest; myocardial infarction; cardiotoxicity caused by compounds such as drugs (e.g., doxorubicin, herceptin, thioridazine and cisapride); cardiac damage due to parasitic infection (bacteria, fungi, rickettsiae, and viruses, e.g., syphilis, chronic Trypanosoma cruzi infection); fulminant cardiac amyloidosis; heart surgery; heart transplantation; traumatic cardiac injury (e.g., penetrating or blunt cardiac injury, and aortic valve rupture), surgical repair of a thoracic aortic aneurysm; a suprarenal aortic aneurysm; cardiogenic shock due to myocardial infarction or cardiac failure; neurogenic shock and anaphylaxis.

“Subject” includes living organisms such as humans, monkeys, cows, sheep, horses, pigs, cattle, goats, dogs, cats, mice, rats, cultured cells therefrom, and transgenic species thereof In a preferred embodiment, the subject is a human. Administration of the compositions of the present invention to a subject to be treated can be carried out using known procedures, at dosages and for periods of time effective to treat the condition in the subject. An effective amount of the therapeutic compound necessary to achieve a therapeutic effect may vary according to factors such as the age, sex, and weight of the subject, and the ability of the therapeutic compound to treat the foreign agents in the subject. Dosage regimens can be adjusted to provide the optimum therapeutic response. For example, several divided doses may be administered daily or the dose may be proportionally reduced as indicated by the exigencies of the therapeutic situation.

“Substantially pure” includes compounds, e.g., drugs, proteins or polypeptides that have been separated from components which naturally accompany it. Typically, a compound is substantially pure when at least 10%, more preferably at least 20%, more preferably at least 50%, more preferably at least 60%, more preferably at least 75%, more preferably at least 90%, and most preferably at least 99% of the total material (by volume, by wet or dry weight, or by mole percent or mole fraction) in a sample is the compound of interest. Purity can be measured by any appropriate method, e.g., in the case of polypeptides by column chromatography, gel electrophoresis or HPLC analysis. A compound, e.g., a protein, is also substantially purified when it is essentially free of naturally associated components or when it is separated from the native contaminants which accompany it in its natural state. Included within the meaning of the term “substantially pure” are compounds, such as proteins or polypeptides, which are homogeneously pure, for example, where at least 95% of the total protein (by volume, by wet or dry weight, or by mole percent or mole fraction) in a sample is the protein or polypeptide of interest.

“Administering” includes routes of administration which allow the compositions of the invention to perform their intended function, e.g., treating or preventing cardiac injury caused by hypoxia or ischemia. A variety of routes of administration are possible including, but not necessarily limited to parenteral (e.g., intravenous, intraarterial, intramuscular, subcutaneous injection), oral (e.g., dietary), topical, nasal, rectal, or via slow releasing microcarriers depending on the disease or condition to be treated. Oral, parenteral and intravenous administration are preferred modes of administration. Formulation of the compound to be administered will vary according to the route of administration selected (e.g., solution, emulsion, gels, aerosols, capsule). An appropriate composition comprising the compound to be administered can be prepared in a physiologically acceptable vehicle or carrier and optional adjuvants and preservatives. For solutions or emulsions, suitable carriers include, for example, aqueous or alcoholic/aqueous solutions, emulsions or suspensions, including saline and buffered media, sterile water, creams, ointments, lotions, oils, pastes and solid carriers. Parenteral vehicles can include sodium chloride solution, Ringer's dextrose, dextrose and sodium chloride, lactated Ringer's or fixed oils. Intravenous vehicles can include various additives, preservatives, or fluid, nutrient or electrolyte replenishers (See generally, Remington's Pharmaceutical Science, 16th Edition, Mack, Ed. (1980)).

“Effective amount” includes those amounts of erythropoietin which allow it to perform its intended function, e.g., treating or preventing, partially or totally, cardiac injury caused by hypoxia or ischemia as described herein. The effective amount will depend upon a number of factors, including biological activity, age, body weight, sex, general health, severity of the condition to be treated, as well as appropriate pharmacolinetic properties. For example, dosages of the active substance may be from about 0.01 mg/kg/day to about 500 mg/kg/day, advantageously from about 0.1 mg/kg/day to about 100 mg/kg/day. A therapeutically effective amount of the active substance can be administered by an appropriate route in a single dose or multiple doses. Further, the dosages of the active substance can be proportionally increased or decreased as indicated by the exigencies of the therapeutic or prophylactic situation.

“Specific binding” or “specifically binds” includes proteins, such as an antibody which recognizes and binds an erythropoietin or a ligand thereof, but does not substantially recognize or bind other molecules in a sample.

“Pharmaceutically acceptable carrier” includes any and all solvents, dispersion media, coatings, antibacterial and antifungal agents, isotonic and absorption delaying agents, and the like which are compatible with the activity of the compound and are physiologically acceptable to the subject. An example of a pharmaceutically acceptable carrier is buffered normal saline (0.15M NaCl). The use of such media and agents for pharmaceutically active substances is well known in the art. Except insofar as any conventional media or agent is incompatible with the therapeutic compound, use thereof in the compositions suitable for pharmaceutical administration is contemplated. Supplementary active compounds can also be incorporated into the compositions.

“Pharmaceutically acceptable esters” includes relatively non-toxic, esterified products of therapeutic compounds of the invention. These esters can be prepared in situ during the final isolation and purification of the therapeutic compounds or by separately reacting the purified therapeutic compound in its free acid form or hydroxyl with a suitable esterifying agent; either of which are methods known to those skilled in the art. Acids can be converted into esters according to methods well known to one of ordinary skill in the art, e.g., via treatment with an alcohol in the presence of a catalyst.

“Additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” which may be included in the pharmaceutical compositions of the invention are known in the art and described, e.g., in Remington 's Pharmaceutical Sciences.

“Unit dose” includes a discrete amount of the pharmaceutical composition comprising a predetermined amount of the active ingredient.

“Protective mechanisms” include biological events and pathways that inhibit or reduce cellular damage caused by oxidative and/or nitrosative stress.

The invention relates in part to methods of treating or preventing myocardial oxidative stress, such as is caused by hypoxia or ischemia, in a subject. This is done by administering to a subject in need thereof erythropoietin which modulates myocardial oxidative stress such that the myocardial cells which are the target of the oxidative stress are protected from cell death. The cell death may be due, e.g., to necrosis or apoptosis.

The invention relates in part to methods of treating or preventing myocardial nitrosative stress, such as is caused by drugs, infection, inflammation, hypoxia, ischemia or other causes, in a subject. This is done by administering to a subject in need thereof erythropoietin which modulates myocardial nitrosative stress such that the myocardial cells which are the target of the nitrosative stress are protected from cell death. The cell death may be due, e.g., to necrosis or apoptosis.

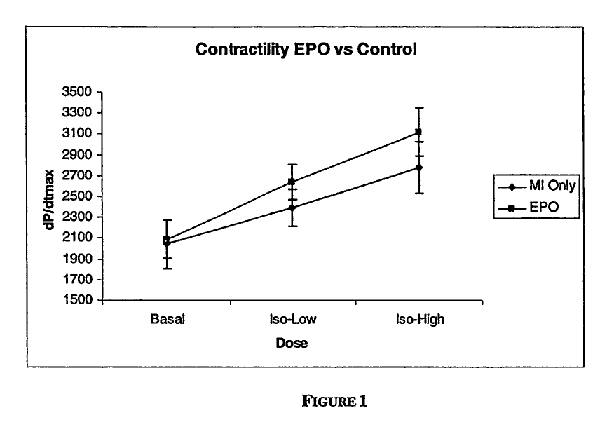

The method by which erythropoietin treatment results in cardioprotection is thought to be independent from erythropoietin's effects on red blood cell numbers (See Example 1; FIGS. 1-3). Therefore, the erythropoietin can be given at such a concentration that the subject's hematocrit level is not substantially increased. A substantial increase in hematocrit would be an increase of about 10% or greater. Methods for measuring the hematocrit in a subject are known in the art, and include centrifugation, automated cell counting, and spectroscopy. Alternatively, the erythropoietin can be given at such a concentration that red blood cell production is not substantially increased in subject. The present invention also encompasses methods in which erythropoietin is given at a concentration that increases cardioprotective mechanisms without substantially increasing a subject's hematocrit level. Non-limiting examples of detectable cardioprotective mechanisms include the Akt kinase pathway (including, e.g., signaling to endothelial nitric oxide synthase) and the ERK kinase (e.g., MEK1) pathway. Alternatively, the erythropoietin is given at the lowest concentration wherein the erythropoietin polypeptide binds to the erythropoietin receptor (EPO-R). Methods for measuring the binding of erythropoietin to EPO-R in vivo or ex vivo are known in the art, including laser-scanning imaging, radio-ligand binding studies, flow cytometry, and agonist/antagonist studies.

Further, erythropoietin can be given for a time period in which the hematocrit of the subject is not increased. Generally, this time period will be about four to five days, but may be longer or shorter as needed for treatment, and erythropoietin may be administered one or more times per day throughout the duration of treatment. Therefore, the methods of this invention are useful as therapeutic and/or protective treatments for subjects (e.g., humans) suffering from or at risk of cardiac injury, for whom addition of erythropoietin such that the hematocrit of the patients is increased elevates the risk of adverse cardiac events. (See Besarab et al. (1998) N. Eng. J. Med. 339: 584). Therefore, the methods of this invention are particularly useful in clinical situations in which it is desirable to treat or prevent a cardiac injury, but that elevation of a patient's hematocrit levels will increase the risk of mortality and/or morbidity.

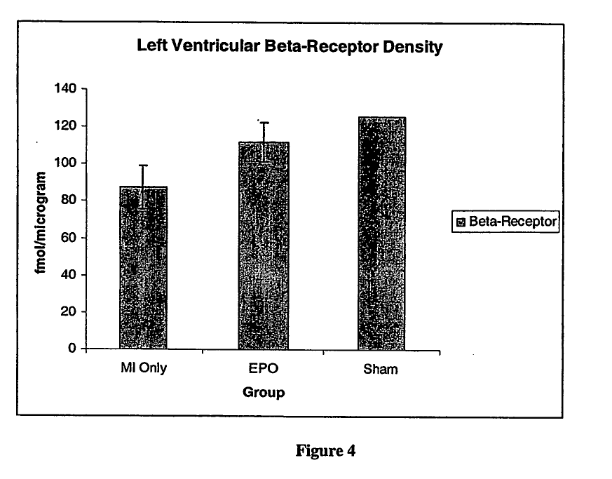

In subjects suffering from myocardial infarction, erythropoietin treatment results in an increase in the density of the beta adrenergic receptor (also known as beta receptor) on the surface of the left ventricle. A decrease in the concentration or activity of beta receptors, such as is known to occur during cardiac injury, requires a corresponding increase in the dosage compounds used as beta receptor agonists (e.g., dopamine, dobutamine, isoprel, and norepinephrine). This dosage increase in turn leads to a loss of tolerance due to over-stimulation, which decreases the effectivity of the administered compounds. Since erythropoietin treatment results in an increase in the density of the beta adrenergic receptors (See Example 2; FIG. 4), it is useful to prevent loss of tolerance and to increase effectivity of administered beta receptor agonists.

Erythropoietin increases the density of beta adrenergic receptors via one or more cell signaling pathways. Increasing the density and/or activity of beta adrenergic receptors by administration of erythropoietin is useful to prevent or treat cardiac injury or defects in heart rhythm (e.g., tachycardia, bradycardia from any cause, and carotid hypersensitivity, such as due to an autonomic dysfunction). Erythropoietin administration is also useful to increase the sensitivity of cardiac tissue to anti-arrhythmic compounds and cardiac contractility enhancing compounds.

Administration of the compounds of the invention may be done where clinically necessary or desirable, e.g., prior to ischemia, at the onset of ischemia, or at one or more times following the onset of ischemia.

The erythropoietin can be obtained by natural sources (e.g. urinary erythropoietin; See U.S. Pat. No. 3,865,801). The purification of human urinary erythropoietin by Miyake et al. in J. Biol. Chem., 252, 5558 (1977), used, as starting material, urine from aplastic anemic individuals. Naturally-occurring human erythropoietin is first translated to a 166 amino acid-containing polypeptide chain with arginine 166. In a postranslational modification arginine 166 is cleaved by a carboxypeptidase. The molecular weight of the polypeptide chain of human erythropoietin without the sugar moieties is 18,236 Da. In the intact erythropoietin molecule, approximately 40% of the molecular weight is accounted for by the carbohydrate groups (Sasaki, H, Bothner, B, Dell, A and Fukuda, M (1987) J. Biol. Chem. 262: 12059).

The identification, cloning, and expression of genes encoding erythropoietin are described in Egrie et al. (1986) Immunobiol. 72: 213-224 and U.S. Pat. No. 4,703,008. A description of the purification of recombinant erythropoietin from cell medium that supported the growth of mammalian cells containing recombinant erythropoietin plasmids for example, is included in U.S. Pat. No. 4,667,016.

Instead of the recombinant erythropoietin protein, modifications of said protein having a higher or lower molecular weight than 34,000 Da (molecular weight of urinary erythropoietin), isoforms of the enzyme or proteins with different glycosylation may also be used. The isoforms of urinary derived human erythropoietin are different than the isoforms of recombinant erythropoietin. Moreover, in principle, those proteins derived from the amino acid sequence of natural erythropoietin with a length of 166 amino acids by way of deletions, substitutions or extensions are also possible. Essentially, such proteins have physiological properties comparable to recombinant erythropoietin. In particular, such proteins have biological properties inducing the bone marrow to increase the production of reticulocytes and red blood cells and/or to increase hemoglobin synthesis or iron absorption. Instead of these proteins, low molecular weight substances may also be used, which are referred to as erythropoietin mimetics and bind to the same biological receptor. Preferably, these miimetics may also be administered by the oral route. The amount of such proteins or mimetics to be administered is determined by comparing the biological activities of erythropoietin and said active substances.

Erythropoietin-like polypeptides are also encompassed by the present invention, including, e.g., darbepoietin (from Amgen; also known as Aranesp and novel erthyropoiesis stimulating protein (NESP)). Administration of darbepoietin for use in the present invention includes subcutaneous or intravenous administration at about 0.5 micrograms/kg once a week.

The invention encompasses the preparation and use of pharmaceutical compositions comprising a compound, such as erythropoietin, useful for the prevention or reduction of hypoxic/ischemic cardiac injury as an active ingredient. Such a pharmaceutical composition may consist of the active ingredient alone, in a form suitable for administration to a subject, or the pharmaceutical composition may comprise the active ingredient and one or more pharmaceutically acceptable carriers, one or more additional ingredients, or some combination of these. The active ingredient may be present in the pharmaceutical composition in the form of a pharmaceutically acceptable ester or salt, such as in combination with a physiologically-acceptable cation or anion, as is well known in the art. Further, the erythropoietin may contain pharmacologically acceptable additives (e.g., carrier, excipient and diluent), stabilizers or components necessary for formulating preparations, which are generally used for pharmaceutical products, as long as it does not adversely affect the efficacy of the preparation, e.g., in decreasing or inhibiting ischemia or reperfusion injury.

Examples of anti-arrhythmic compounds include, e.g., adenosine, amiodarone, bretylium, disopyramide, flecainide, lignocaine, mexiletine and propafenone.

Examples of contractility enhancing compounds include cardiac glycosides such as digoxin and digitoxin, sympathomimetic amines such as dobutamine and dopamine, phosphodiesterase inhibitors such as amrinone and milrinone, compounds that increase sarcoplasmic reticulum Ca²⁺-ATPase activity, contractilin, isoproterenol and other compounds.

Examples of additives and stabilizers include saccharides such as monosaccharides (e.g., glucose and fructose), disaccharides (e.g., sucrose, lactose and maltose) and sugar alcohols (e.g., mannitol and sorbitol); organic acids such as citric acid, maleic acid and tartaric acid and salts thereof (e.g., sodium salt, potassium salt and calcium salt); amino acids such as glycine, aspartic acid and glutamic acid and salts thereof (e.g., sodium, calcium or potassium salt); surfactants such as polyethylene glycol, polyoxyethylene-polyoxypropylene copolymer and polyoxyethylenesorbitan fatty acid ester; heparin; and albumin.

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

Although the descriptions of pharmaceutical compositions provided herein are principally directed to pharmaceutical compositions that are suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions of the invention is contemplated include, but are not limited to, humans and other primates.

Pharmaceutical compositions that are useful in the methods of the invention maybe prepared, packaged, or sold in formulations suitable for oral, rectal, vaginal, parenteral, topical, pulmonary (See e.g., U.S. Pat. No. 5,354,934), intranasal, buccal, ophthalmic, or another route of administration. The preferred mode is intravenous administration.

The erythropoietin and the above-mentioned ingredients are admixed as appropriate to give powder, granule, tablet, capsule, syrup, injection and the like. Other contemplated formulations include projected nanoparticles, liposomal preparations, resealed erythrocytes containing the active ingredient, and immunologically-based formulations.

A pharmaceutical composition of the invention may be prepared, packaged, or sold in bulk, as a single unit dose, or as a plurality of single unit doses. The amount of the active ingredient is generally equal to the dosage of the active ingredient, which would be administered to a subject, or a convenient fraction of such a dosage such as, for example, one-half or one-third of such a dosage.

The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient.

In addition to the active ingredient, a pharmaceutical composition of the invention may further comprise one or more additional pharmaceutically active agents.

Particularly contemplated additional agents include anti-emetics and scavengers such as cyanide and cyanate scavengers. Controlled- or sustained-release formulations of a pharmaceutical composition of the invention may be made using conventional technology.

A formulation of a pharmaceutical composition of the invention suitable for oral administration may be prepared, packaged, or sold in the form of a discrete solid dose unit including, but not limited to, a tablet, a hard or soft capsule, a cachet, a troche, or a lozenge, each containing a predetermined amount of the active ingredient. Other formulations suitable for oral administration include, but are not limited to, a powdered or granular formulation, an aqueous or oily suspension, an aqueous or oily solution, or an emulsion.

A tablet comprising the active ingredient may, for example, be made by compressing or molding the active ingredient, optionally with one or more additional ingredients. Compressed tablets may be prepared by compressing, in a suitable device, the active ingredient in a free-flowing form such as a powder or granular preparation, optionally mixed with one or more of a binder, a lubricant, an excipient, a surface active agent, and a dispersing agent. Molded tablets may be made by molding, in a suitable device, a mixture of the active ingredient, a pharmaceutically acceptable carrier, and at least sufficient liquid to moisten the mixture. Pharmaceutically acceptable excipients used in the manufacture of tablets include, but are not limited to, inert diluents, granulating and disintegrating agents, binding agents, and lubricating agents. Known dispersing agents include potato starch and sodium starch glycollate. Known surface active agents include sodium lauryl sulfate. Known diluents include calcium carbonate, sodium carbonate, lactose, microcrystalline cellulose, calcium phosphate, calcium hydrogen phosphate, and sodium phosphate. Known granulating and disintegrating agents include corn starch and alginic acid. Known binding agents include gelatin, acacia, pre-gelatinized maize starch, polyvinylpyrrolidone, and hydroxypropyl methylcellulose. Known lubricating agents include magnesium stearate, stearic acid, silica, and talc.

Tablets may be non-coated or they may be coated using known methods to achieve delayed disintegration in the gastrointestinal tract of a subject, thereby providing sustained release and absorption of the active ingredient. By way of example, a material such as glyceryl monostearate or glyceryl distearate may be used to coat tablets. Further by way of example, tablets may be coated using methods described in, e.g., U.S. Pat. Nos. 4,256,108; 4,160,452; and 4,265,874 to form osmotically-controlled release tablets. Tablets may further comprise a sweetening agent, a flavoring agent, a coloring agent, a preservative, or some combination of these in order to provide pharmaceutically elegant and palatable preparation.

Hard capsules comprising the active ingredient may be made using a physiologically degradable composition, such as gelatin. Such hard capsules comprise the active ingredient, and may further comprise additional ingredients including, for example, an inert solid diluent such as calcium carbonate, calcium phosphate, or kaolin.

Soft gelatin capsules comprising the active ingredient may be made using a physiologically degradable composition, such as gelatin. Such soft capsules comprise the active ingredient, which may be mixed with water or an oil medium such as peanut oil, liquid paraffin, or olive oil.

Liquid formulations of a pharmaceutical composition of the invention which are suitable for oral administration may be prepared, packaged, and sold either in liquid form or in the form of a dry product intended for reconstitution with water or another suitable vehicle prior to use.

Liquid suspensions may be prepared using conventional methods to achieve suspension of the active ingredient in an aqueous or oily vehicle. Aqueous vehicles include, for example, water and isotonic saline. Oily vehicles include, for example, almond oil, oily esters, ethyl alcohol, vegetable oils such as arachis, olive, sesame, or coconut oil, fractionated vegetable oils, and mineral oils such as liquid paraffin. Liquid suspensions may further comprise one or more additional ingredients including, but not limited to, suspending agents, dispersing or wetting agents, emulsifying agents, demulcents, preservatives, buffers, salts, flavorings, coloring agents, and sweetening agents. Oily suspensions may further comprise a thickening agent. Known suspending agents include, but are not limited to, sorbitol syrup, hydrogenated edible fats, sodium alginate, polyvinylpyrrolidone, gum tragacanth, gum acacia, and cellulose derivatives such as sodium carboxymethylcellulose, methylcellulose, hydroxypropylmethylcellulose. Known dispersing or wetting agents include naturally-occurring phosphatides such as lecithin, condensation products of an alkylene oxide with a fatty acid, with a long chain aliphatic alcohol, with a partial ester derived from a fatty acid and a hexitol, or with a partial ester derived from a fatty acid and a hexitol anhydride (e.g., polyoxyethylene stearate, heptadecaethyleneoxycetanol, polyoxyethylene sorbitol monooleate, and polyoxyethylene sorbitan monooleate, respectively). Known emulsifying agents include lecithin and acacia. Known preservatives include methyl, ethyl, or n-propyl-para-hydroxybenzoates, ascorbic acid, and sorbic acid. Known sweetening agents include glycerol, propylene glycol, sorbitol, sucrose, and saccharin. Known thickening agents for oily suspensions include, for example, beeswax, hard paraffin, and cetyl alcohol.

Liquid solutions of the active ingredient in aqueous or oily solvents may be prepared in substantially the same manner as liquid suspensions, the primary difference being that the active ingredient is dissolved, rather than suspended in the solvent. Liquid solutions of the pharmaceutical composition of the invention may comprise each of the components described with regard to liquid suspensions, it being understood that suspending agents will not necessarily aid dissolution of the active ingredient in the solvent. Aqueous solvents include, for example, water and isotonic saline. Oily solvents include, for example, almond oil, oily esters, ethyl alcohol, vegetable oils such as arachis, olive, sesame, or coconut oil, fractionated vegetable oils, and mineral oils such as liquid paraffin.

Powdered and granular formulations of a pharmaceutical preparation of the invention may be prepared using known methods. Such formulations may be administered directly to a subject, used, for example, to form tablets, to fill capsules, or to prepare an aqueous or oily suspension or solution by addition of an aqueous or oily vehicle thereto. Each of these formulations may further comprise one or more of dispersing or wetting agent, a suspending agent, and a preservative. Additional excipients, such as fillers and sweetening, flavoring, or coloring agents, may also be included in these formulations.

A pharmaceutical composition of the invention may also be prepared, packaged, or sold in the form of oil-in-water emulsion or a water-in-oil emulsion. The oily phase may be a vegetable oil such as olive or arachis oil, a mineral oil such as liquid paraffin, or a combination of these. Such compositions may further comprise one or more emulsifying agents such as naturally occurring gums such as gum acacia or gum tragacanth, naturally-occurring phosphatides such as soybean or lecithin phosphatide, esters or partial esters derived from combinations of fatty acids and hexitol anhydrides such as sorbitan monooleate, and condensation products of such partial esters with ethylene oxide such as polyoxyethylene sorbitan monooleate. These emulsions may also contain additional ingredients including, for example, sweetening or flavoring agents.

A pharmaceutical composition of the invention may be prepared, packaged, or sold in a formulation suitable for rectal administration. Such a composition may be in the form of, for example, a suppository, a retention enema preparation, and a solution for rectal or colonic irrigation.

Suppository formulations may be made by combining the active ingredient with a non-irritating pharmaceutically acceptable excipient which is solid at ordinary room temperature (i.e., about 20° C.) and which is liquid at the rectal temperature of the subject (i.e., about 37° C. in a healthy human). Suitable pharmaceutically acceptable excipients include, but are not limited to, cocoa butter, polyethylene glycols, and various glycerides. Suppository formulations may further comprise various additional ingredients including, but not limited to, antioxidants and preservatives.

Retention enema preparations or solutions for rectal or colonic irrigation may be made by combining the active ingredient with a pharmaceutically acceptable liquid carrier. As is well known in the art, enema preparations may be administered using, and may be packaged within, a delivery device adapted to the rectal anatomy of the subject. Enema preparations may further comprise various additional ingredients including, but not limited to, antioxidants and preservatives.

A pharmaceutical composition of the invention may be prepared, packaged, or sold in a formulation suitable for vaginal administration. Such a composition may be in the form of, for example, a suppository, an impregnated or coated vaginally-insertable material such as a tampon, a douche preparation, a gel or cream or solution for vaginal irrigation.

Methods for impregnating or coating a material with a chemical composition are known in the art, and include, but are not limited to methods of depositing or binding a chemical composition onto a surface, methods of incorporating a chemical composition into the structure of a material during the synthesis of the material (i.e., such as with a physiologically degradable material), and methods of absorbing an aqueous or oily solution or suspension into an absorbent material, with or without subsequent drying.

Douche preparations or solutions for vaginal irrigation may be made by combining the active ingredient with a pharmaceutically acceptable liquid carrier. As is well known in the art, douche preparations may be administered using, and may be packaged within, a delivery device adapted to the vaginal anatomy of the subject.

Douche preparations may further comprise various additional ingredients including, but not limited to, antioxidants, antibiotics, antifungal agents, and preservatives.

Additional delivery methods for administration of compounds include a drug delivery device, such as that described in U.S. Pat. No. 5,928,195.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intraperitoneal, intramuscular, intrasternal injection, and kidney dialytic infusion techniques.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi-dose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In one embodiment of a formulation for parenteral administration, the active ingredient is provided in dry (i.e., powder or granular) form for reconstitution with a suitable vehicle (e.g., sterile pyrogen-free water) prior to parenteral administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or diglycerides. Other parentally-administrable formulations that are useful include those, which comprise the active ingredient in microcrystalline form, in a liposomal preparation, or as a component of a biodegradable polymer systems. Compositions for sustained release or implantation may comprise pharmaceutically acceptable polymeric or hydrophobic materials such as an emulsion, an ion exchange resin, a sparingly soluble polymer, or a sparingly soluble salt.

Formulations suitable for topical administration include, but are not limited to, liquid or semi-liquid preparations such as liniments, lotions, oil-in-water or water-in-oil emulsions such as creams, ointments or pastes, and solutions or suspensions. Topically-administrable formulations may, for example, comprise from about 1% to about 10% (w/w) active ingredient, although the concentration of the active ingredient may be as high as the solubility limit of the active ingredient in the solvent. Formulations for topical administration may further comprise one or more of the additional ingredients described herein.

A pharmaceutical composition of the invention may be prepared, packaged, or sold in a formulation suitable for pulmonary administration via the buccal cavity. Such a formulation may comprise dry particles which comprise the active ingredient and which have a diameter in the range from about 0.5 to about 7 nanometers, and preferably from about 1 to about 6 nanometers. Such compositions are conveniently in the form of dry powders for administration using a device comprising a dry powder reservoir to which a stream of propellant may be directed to disperse the powder or using a self-propelling solvent/powder-dispensing container such as a device comprising the active ingredient dissolved or suspended in a low-boiling propellant in a sealed container. Preferably, such powders comprise particles wherein at least 98% of the particles by weight have a diameter greater than 0.5 nanometers and at least 95% of the particles by number have a diameter less than 7 nanometers. More preferably, at least 95% of the particles by weight have a diameter greater than 1 nanometer and at least 90% of the particles by number have a diameter less than 6 nanometers. Dry powder compositions preferably include a solid fine powder diluent such as sugar and are conveniently provided in a unit dose form.

Low boiling propellants generally include liquid propellants having a boiling point of below 65° F. at atmospheric pressure. Generally the propellant may constitute 50 to 99.9% (w/w) of the composition, and the active ingredient may constitute 0.1 to 20% (w/w) of the composition. The propellant may further comprise additional ingredients such as a liquid non-ionic or solid anionic surfactant or a solid diluent (preferably having a particle size of the same order as particles comprising the active ingredient).

Pharmaceutical compositions of the invention formulated for pulmonary delivery may also provide the active ingredient in the form of droplets of a solution or suspension. Such formulations may be prepared, packaged, or sold as aqueous or dilute alcoholic solutions or suspensions, optionally sterile, comprising the active ingredient, and may conveniently be administered using any nebulization or atomization device. Such formulations may fer comprise one or more additional ingredients including, but not limited to, a flavoring agent such as saccharin sodium, a volatile oil, a buffering agent, a surface active agent, or a preservative such as methylhydroxybenzoate. The droplets provided by this route of administration preferably have an average diameter in the range from about 0.1 to about 200 nanometers.

The formulations described herein as being useful for pulmonary delivery are also useful for intranasal delivery of a pharmaceutical composition of the invention.

Another formulation suitable for intranasal administration is a coarse powder comprising the active ingredient and having an average particle from about 0.2 to 500 micrometers. Such a formulation is administered in the manner in which snuff is taken i.e., by rapid inhalation through the nasal passage from a container of the powder held close to the nose.

Formulations suitable for nasal administration may, for example, comprise from about as little as 0.1% (w/w) and as much as 100% (w/w) of the active ingredient, and may frrther comprise one or more of the additional ingredients described herein.

A pharmaceutical composition of the invention may be prepared, packaged, or sold in a formulation suitable for buccal administration. Such formulations may, for example, be in the form of tablets or lozenges made using conventional methods, and may, for example, 0.1 to 20% (w/w) active ingredient, the balance comprising an orally dissolvable or degradable composition and, optionally, one or more of the additional ingredients described herein. Alternately, formulations suitable for buccal administration may comprise a powder or an aerosolized or atomized solution or suspension comprising the active ingredient. Such powdered, aerosolized, or aerosolized formulations, when dispersed, preferably have an average particle or droplet size in the range from about 0.1 to about 200 nanometers, and may further comprise one or more of the additional ingredients described herein.

A pharmaceutical composition of the invention may be prepared, packaged, or sold in a formulation suitable for ophthalmic administration. Such formulations may, for example, be in the form of eye drops including, for example, a 0.1-1.0% (w/w) solution or suspension of the active ingredient in an aqueous or oily liquid carrier. Such drops may further comprise buffering agents, salts, or one or more other of the additional ingredients described herein. Other ophthalmalmically-administrable formulations that are useful include those, which comprise the active ingredient in microcrystalline form or in a liposomal preparation.

The mixture of erythropoietin and pharmacologically acceptable additives is preferably prepared as a lyophilized product, and dissolved when in use. Such preparation can be prepared into a solution containing about 0.01-100.0 mg/ml of erythropoietin, by dissolving same in distilled water for injection or sterile purified water. More preferably, it is adjusted to have a physiologically isotonic salt concentration and a physiologically desirable pH value (pH 6-8).

Erythropoietin serum concentration is normally within the range of 5-50 mU/ml. For patients suffering from chronic renal failure or other conditions involving anemia, erythropoietin is generally administered either subcutaneously or intravenously at a concentration of 50-100 U/kg (a dose of 3,000-7,000 U) three times per week, or once one week prior to surgery. While the dose is appropriately determined depending on symptom, body weight, sex, animal species and the like, it is generally assumed that treatment options holding the blood concentration at about 1-100 mU/ml will be preferred. This plasma concentration may be achieved through administration of one to several doses a day. When erythropoietin is to be administered to a subject, 0.1 ng to 10 mg/kg body weight (e.g. 1 ng to 1 mg/kg body weight) of erythropoietin can be given intravenously.

The compound may be administered to an animal as frequently as several times daily, or it may be administered less frequently, such as once a day, once a week, once every two weeks, once a month, or even lees frequently, such as once every several months or even once a year or less. The frequency of the dose will be readily apparent to the skilled artisan and will depend upon any number of factors, such as, but not limited to, the type and severity of the disease being treated, the type and age of the animal, etc.

### EXAMPLES

These Examples are provided for the purpose of illustration only and the invention should in no way be construed as being limited to these Examples, but rather should be construed to encompass any and all variations which become evident as a result of the teaching provided herein.

### Example 1

### In Vivo Studies of Erythropoietin Preservation of Cardiac Contractility

A coronary artery ligation model was used to demonstrate the protective effect of erythropoietin in the absence of an increase in hematocrit. Animals used in this study were adult male New Zealand White rabbits (3-5 kg, generally 4 kg). Animals were housed under standard conditions and allowed to feed ad lib. The Animal Care and Use Committee of Duke University approved all procedures performed in accordance with the regulations adopted by the National Institutes of Health. A myocardial infarction (MI) was produced via the ligation of a marginal branch of the left circumflex coronary artery (LCx) using 5-0 prolene suture (See e.g., Maurice et al. Am J Physiol. 276:H1853-H1860, 1999; Shah et al., Circulation 103:1311-1316, 2001). Rabbits were anesthetized with a mixture of ketamine (30 mg/kg) and acepromazine (0.5 mg/kg), intubated, and mechanically ventilated. A left thoracotomy was performed through the 3^{rd} or 4^{th} intercostal space, and the large marginal branch of the LCx was identified and ligated with a 5-0 Prolene suture. A control group included sham operated animals in which only a thoracotomy and pericardiotomy were performed. Anatomic closure was performed, the chest was evacuated of residual air using a 14-gauge angiocatheter attached to a syringe, and the animal was extubated when able to breathe spontaneously. Animals were allowed to recover and returned to their cages when awake and responsive. For characterization of infarction size, hearts were dissected after euthanasia and the aortic root was cannulated. Each heart was rinsed with 40 cc of normal saline followed by 40 cc of triphenyltetrazolium chloride (SIGMA 8) at 37° C. The heart was then disected at the right ventricular free wall and both atria. The heart was sectioned in 2-3 mm segments from apex to atrio-ventricular groove in a transverse fashion. Each segment was weighed, recorded and placed in formalin. After six hours the specimen was digitally photographed in a camera mount to normalize specimen-to-lens distance. Each photograph was then appended to Adobe Photoshop (Adobe A) to measure pixel density of infarcted versus non infarcted areas. The percentage of infarction of each slide was multiplied by the mass of each specimen. The sum of all specimen percentages resulted in an overall percentage of infarction in each animal.

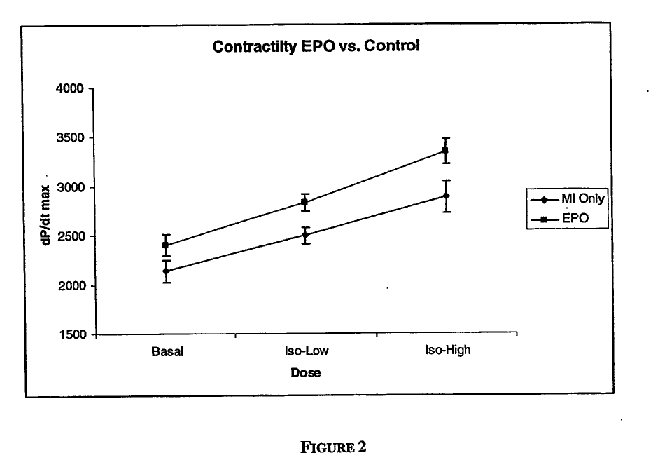

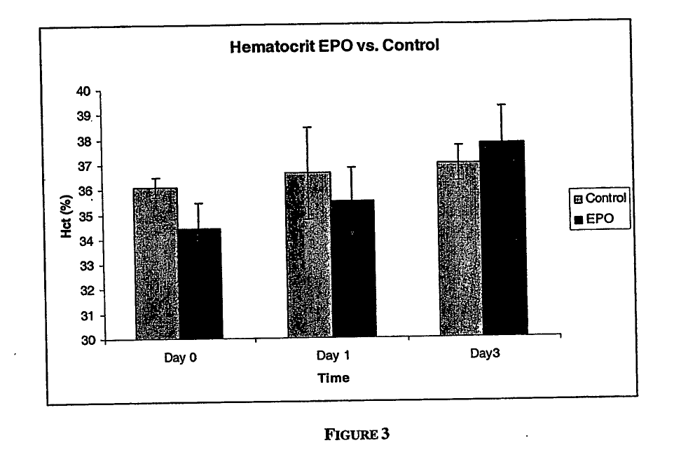

To measure in vivo hemodynamic data in conscious animals, rabbits were lightly sedated with ketamine (30 mg/kg) and acepromazine (0.05 mg/kg). The right carotid artery was then exposed and a 2.5 Fr micromanometer (Millar Instruments) was advanced into the LV cavity to record hemodynamics. FIG. 1 demonstrates an increase in cardiac contractility when erythropoietin is administered 5 minutes after myocardial ischemia induced by suture ligation of the large branch of the left circumflex artery. FIG. 2 demonstrates an increase in cardiac contractility when erythropoietin is administered 24 hous prior to myocardial ischemia induced by suture ligation of the large branch of the left circumflex artery. FIG. 3 demonstrates that hematocrit levels are not acutely increased four days after administration of 5,000 U/kg of erythropoietin. Similarly, administration of 1,000 U/kg of erythropoietin is cardioprotective as measured by increased cardiac contractility, but does not acutely increase hematocrit levels four days after administration. Administration of lower concentrations of erythropoietin (e.g., 750 U/kg, 500 U/kg, 250 U/kg, 100 U/kg, 50 U/kg, or lower levels) are also encompassed by the present invention. Serial hematocrits were obtained on the day of erythropoietin administration and then serially for a period of four days.

### Example 2

### In Vivo Studies of Erythropoietin Preservation of Left Ventricular Beta-receptor Density Following Myocardial Infarction

A beta agonist receptor ligand binding assay was used to demonstrate the maintenance of left ventricular beta receptor levels following administration of erythropoietin. Myocardial membranes were prepared from frozen hearts (See, e.g., Maurice et al. Am J Physiol. 276:H1853-H1860, 1999). Final purified cardiac membranes were suspended at a concentration of 1-2 mg/ml and receptor binding was performed using the nonselective βAR ligand [¹²⁵I] cyanopindolol. Nonspecific binding was determined in the presence of 20 μM alprenolol. All assays were performed in triplicate, and receptor density (measured in fmoles) was normalized to mg of membrane protein. As shown in FIG. 4, myocardial infarction causes a reduction in cardiac beta receptor density, which is mitigated by treatment with erytlropoietin. Administration of any concentration of erythropoietin that does not increase hematocrit levels is useful to inhibit or decrease the reduction in cardiac beta receptor density caused by cardiac events, such as myocardial infarction. Specifically, erythropoietin can be administered at concentrations of 5,000 U/kg, 1,000 U/kg, 750 U/kg, 500 U/kg, 250 U/kg, 100 U/kg, 50 U/kg, or lower levels).

### Equivalents

Those skilled in the art will recognize, or be able to ascertain using no more than routine experimentation, numerous equivalents to the specific procedures described herein. Such equivalents are considered to be within the scope of the present invention and are covered by the following claims. Various substitutions, alterations, and modifications may be made to the invention without departing from the spirit and scope of the invention as defined by the claims. Other aspects, advantages, and modifications are within the scope of the invention. The contents of all references, issued patents, and published patent applications cited throughout this application are hereby incorporated by reference.

What is claimed is:

1. A method of treating or preventing myocardial oxidative stress in a subject, comprising administering erythropoietin to a subject in need thereof at a concentration that does not increase the hematocrit in said subject.

2. The method of claim 1, wherein said myocardial oxidative stress is caused by hypoxia or ischemia.

3. The method of claim 1, wherein said erythropoietin is administered at a concentration of less than about 5,000 U/kg.

4. A method of treating or preventing myocardial oxidative stress in a subject, comprising administering erythropoietin to a subject in need thereof at a concentration that does not induce red blood cell production in said subject.

5. The method of claim 4, wherein said erythropoietin is administered at a concentration of less than about 5,000 U/kg.

6. A method of treating or preventing myocardial oxidative stress in a subject, comprising administering erythropoietin to a subject in need thereof for a time period in which the hematocrit in said subject is not increased.

7. The method of claim 6, wherein said time period is about one week.

8. The method of claim 6, wherein said time period is about four days.

9. A method of treating or preventing myocardial nitrosative stress in a subject, comprising administering erythropoietin to a subject in need thereof at a concentration that does not increase the hematocrit in said subject.

10. The method of claim 9, wherein said myocardial nitrosative stress is caused by drugs, infection, inflammation, hypoxia or ischemia.

11. The method of claim 9, wherein said erythropoietin is administered at a concentration of less than about 5,000 U/kg.

12. A method of treating or preventing myocardial nitrosative stress in a subject, comprising administering erythropoietin to a subject in need thereof at a concentration that does not induce red blood cell production in said subject.

13. The method of claim 12, wherein said erythropoietin is administered at a concentration of less than about 5,000 U/kg.

14. A method of treating or preventing myocardial nitrosative stress in a subject, comprising administering erythropoietin to a subject in need thereof for a time period in which the hematocrit in said subject is not increased.

15. The method of claim 14, wherein said time period is about one week.

16. The method of claim 14, wherein said time period is about four days.

17. A method of modulating a cardioprotective signaling pathway, comprising administering, to a subject in need of cardioprotection, erythropoietin at a concentration that does not induce red blood cell production in said subject.

18. The method of claim 17, wherein said cardioprotective signaling pathway is selected from the group consisting of MAP kinase, PI3 kinase, an insulin-responsive pathway, hormones, ischemia preconditioning, adenosine pathways, ras, JAK/STAT, nitric oxide synthase, hemoxygenase, xanthine oxidase, NADPH oxidase, cytochrome p450, cytochrome p450 reductase, oxigenases, denitrosylases, GSNO reductase, oxygen-carrying proteins, nitric oxide-carrying proteins, and carbon monoxide-carrying proteins.

19. A method of treating or preventing cardiac injury in a subject, comprising administering erythropoietin to a subject in need thereof at a concentration that does not increase the hematocrit in said subject.

20. The method of claim 19, wherein said cardiac injury is caused by hypoxia or ischemia.

21. The method of claim 19, wherein said injury is selected from the group consisting of myocardial infarction, cardiac arrest, ischemia-reperfusion injury, congestive heart failure, cardiotoxicity, cardiac damage due to parasitic infection, fulminant cardiac amyloidosis, heart surgery, heart transplantation, traumatic cardiac injury, surgical repair of a thoracic aortic aneurysm, a suprarenal aortic aneurysm, hemorrhagic shock due to blood loss, cardiogenic shock due to myocardial infarction or cardiac failure, and anaphylaxis.

22. The method of claim 19, wherein said administration is done prior to reperfusion or infarction.

23. The method of claim 19, wherein said administration is done at the onset of reperfusion.

24. The method of claim, 19, wherein said administration is done subsequent to infarction or cardiac damage.

25. The method of claim 19, wherein the subject also suffers from end-stage renal disease or diabetes.

26. A method of treating or preventing cardiac injury in a subject, comprising administering erythropoietin to a subject in need thereof at a concentration that does not induce red blood cell production in said subject.

27. The method of claim 26, wherein said cardiac injury is caused by hypoxia or ischemia.

28. The method of claim 26, wherein said injury is selected from the group consisting of myocardial infarction, cardiac arrest, ischemia-reperfusion injury, congestive heart failure, cardiotoxicity, cardiac damage due to parasitic infection, fulminant cardiac amyloidosis, heart surgery, heart transplantation, traumatic cardiac injury, surgical repair of a thoracic aortic aneurysm, a suprarenal aortic aneurysm, hemorrhagic shock due to blood loss, cardiogenic shock due to myocardial infarction or cardiac failure, anaphylaxis, unstable coronary syndrome, tachycardia and bradycardia.

29. The method of claim 26, wherein said administration is done prior to reperfusion.

30. The method of claim 26, wherein said administration is done at the onset of reperfusion.

31. The method of claim 26, wherein the subject also suffers from end-stage renal disease or diabetes.

32. A method of treating or preventing cardiac injury in a subject, comprising administering erythropoietin to a subject in need thereof for a time period in which the hematocrit in said subject is not increased.

33. The method of claim 32, wherein said time period is about one week.

34. The method of claim 32, wherein said time period is about four days.

35. The method of claim 32, wherein said injury is selected from the group consisting of myocardial infarction, cardiac arrest, ischemia-reperfusion injury, congestive heart failure, cardiotoxicity, cardiac damage due to parasitic infection, fulminant cardiac amyloidosis, heart surgery, heart transplantation, traumatic cardiac injury, surgical repair of a thoracic aortic aneurysm, a suprarenal aortic aneurysm, hemorrhagic shock due to blood loss, cardiogenic shock due to myocardial infarction or cardiac failure, anaphylaxis, unstable coronary syndrome, tachycardia and bradycardia

36. A method of preventing organ or tissue damage during organ or tissue transplantation, comprising administering to an organ donor erythropoietin at a concentration that does not increase the hematocrit in said donor, prior to or concurrent with removal of said organ.

37. The method of claim 36, wherein said organ is the heart.

38. A method of treating heart failure in a subject, comprising treating said subject with erythropoietin at a concentration that does not increase the hematocrit in said subject and a compound selected from the group consisting of anti-platelet drugs, anti-coagulant drugs, anti-thrombotic drugs, anti-oxidants, anti-nitrosants, cholesterol lowering drugs, aspirin and aspirin-related derivatives.

39. A method of treating a survivor of a myocardial infarction, comprising administering erythropoietin at a concentration that does not increase the hematocrit in said survivor, wherein the erythropoietin is administered in a single dose within 3 hours of the myocardial infarction.

40. The method of claim 39, when the erythropoietin is administered in a single dose within 1 hour of the myoc dial infarction.

41. The method of claim 39, wherein the erythropoietin is administered in a single dose within 5 minutes of the myocardial infarction.

42. The method of claim 39, further comprising administering to said survivor a compound selected from the group consisting of an angiotensin-converting enzyme inhibitor, GLP-1, beta blockers, thrombolytics, ADP receptor antagonists, anti-platelet drugs, anti-coagulant drugs, anti-thrombotic drugs, anti-oxidants, anti-nitrosants, cholesterol lowering drugs, aspirin and aspirin-related derivatives.

43. The method of claim 39, wherein the survivor also suffers from end-stage renal failure or diabetes.

44. A method of treating a survivor of a myocardial infarction, comprising administering erythropoietin at a concentration that does not increase the hematocrit in said survivor, wherein the erythropoietin is administered for an extended period of time.

45. The method of claim 44, wherein said administration is continuous.

46. The method of claim 44, further comprising administering to said survivor a compound selected from the group consisting of an angiotensin-converting enzyme inhibitor, GLP-1, beta blockers, thrombolytics, ADP receptor antagonists, anti-platelet drugs, anti-coagulant drugs, anti-thrombotic drugs, anti-oxidants, anti-nitrosants, cholesterol lowering drugs, aspirin and aspirin-related derivatives.

47. The method of claim 44, wherein the survivor also suffers from end-stage renal failure or diabetes.

48. A method of preventing or reducing the severity of ischemia-reperfusion injury in a subject at risk for ischemia-reperfusion injury comprising administering to the subject an amount of erythropoietin at a concentration that does not increase the hematocrit of the subject.

49. The method of claim 48, wherein the subject is at risk for ischemia-reperfusion injury due to undergoing treatment for myocardial infarction, cardiac arrest, ischemia-reperfusion injury, congestive heart failure, cardiotoxicity, cardiac damage due to parasitic infection, fulminant cardiac amyloidosis, heart surgery, heart transplantation, traumatic cardiac injury, surgical repair of a thoracic aortic aneurysm, a suprarenal aortic aneurysm, hemorrhagic shock due to blood loss, cardiogenic shock due to myocardial infarction or cardiac failure, anaphylaxis, a suprarenal aortic aneurysm, liver, kidney, small intestine, or pancreas transplant, hepatic and biliary surgical resections, total or partial pancreatectomy, total and partial gastrectomy, esophagectomy, colorectal surgery, vascular surgery for mesenteric vascular disease, abdominal insufflation during laparoscopic surgical procedures, blunt or penetrating trauma to the abdomen including gun shot wounds, stab wounds or penetrating wounds or blunt abdominal trauma secondary to deceleration injury or motor vehicle accidents and neurogenic shock.

50. The method of claim 48, wherein erythropoietin is administered to the subject prior to undergoing treatment.

51. The method of claim 48, wherein erythropoietin is administered to the subject subsequent to undergoing treatment.

52. A method of pre-conditioning a subject at risk for a cardiac injury due to a surgical procedure, comprising administering to the subject an amount of erythropoietin at a concentration that does not increase the hematocrit of the subject prior to the surgical procedure.

53. The method of claim 52, wherein erythropoietin is administered to the subject at least about once per day for about seven days prior to said surgical procedure.

54. The method of claim 52, wherein erythropoietin is administered to the subject at least about one day prior to said surgical procedure.

55. A method of pre-conditioning a subject at risk for a cardiac injury due to a surgical procedure, comprising administering to the subject an amount of erythropoietin for a time period in which the hematocrit in said subject is not increased.

56. The method of claim 55, wherein said time period is about one week.

57. The method of claim 55, wherein said time period is about four days.

58. The method of claim 55, wherein erythropoietin is administered to the subject at least about once per day for about three days prior to said surgical procedure.

59. The method of claim 55, wherein erythropoietin is administered to the subject at least about one day prior to said surgical procedure.

60. A method of increasing beta-receptor density in a subject suffering from cardiac injury, comprising administering erythropoietin at a concentration that does not increase the hematocrit in said subject.

61. The method of claim 60, wherein said cardiac injury is selected from the group consisting of myocardial infarction and cardiac stunning from ischemia or apoptosis.

62. The method of claim 60, wherein the erythropoietin is administered for an extended period of time.

63. A method of preserving beta-receptor sensitivity in a subject suffering from cardiac injury, comprising administering erythropoietin at a concentration that does not increase the hematocrit in said subject.

64. A method of preserving beta-receptor sensitivity in a subject suffering from cardiac injury, comprising administering erythropoietin at a concentration that does not induce red blood cell production.

65. A method of preventing reduced sensitivity to one or more cardiostimulatory compounds in a subject suffering from or at risk of a cardiac injury, comprising administering erythropoietin at a concentration that does not increase the hematocrit in said subject.

66. The method of claim 65, wherein said cardiostimulatory compounds are selected from the group consisting of anti-arrhythmic compounds and contractility enhancing compounds.

67. The method of claim 65, wherein said cardiostimulatory compounds are selected from the group consisting of dopamine, dobutamine, isoprel, digoxin, digitoxin, and norepinephrine.

68. A method of increasing sensitivity to one or more cardiostimulatory compounds in a subject suffering from or at risk of a cardiac injury, comprising administering erythropoietin at a concentration that does not increase the hematocrit in said subject.

69. The method of claim 68, wherein said cardiostimulatory compounds are selected from the group consisting of anti-arrhythmic compounds and contractility enhancing compounds.

70. The method of claim 68, wherein said cardiostimulatory compounds are selected from the group consisting of dopamine, dobutamine, isoprel, digoxin, digitoxin, and norepinephrine.

71. A method of increasing beta-receptor density in a subject suffering from cardiac injury, comprising administering to the subject an amount of erythropoietin for a time period in which the hematocrit in said subject is not increased.

72. The method of claim 71, wherein said time period is about one week.

73. The method of claim 71, wherein said time period is about four days.
